(19) United States
(12) Patent Application Publication
(10) Pub. No.: US 2023/0066795 A1
(43) Pub. Date: Mar. 2, 2023

(54) PIVC-INTEGRATED HEMOLYSIS-REDUCTION ACCESSORIES FOR DIRECT BLOOD DRAW

(71) Applicant: CareFusion 303, Inc., San Diego, CA (US)

(72) Inventors: Jon K. BURKHOLZ, Salt Lake City, UT (US); Bin WANG, Sandy, UT (US); George MANSOUR, Diamond Bar, CA (US)

(21) Appl. No.: 17/900,844

(22) Filed: Aug. 31, 2022

Related U.S. Application Data
(60) Provisional application No. 63/239,639, filed on Sep. 1, 2021.

(51) Int. Cl. A61B 5/15

(52) U.S. Cl. CPC A61B 5/150221; A61B 5/15003; A61B 5/150992; A61M 25/0097

(57) ABSTRACT

A flow restriction device may include a distal end configured to couple to a catheter assembly, a proximal end configured to couple to a fluid collection device, and a body extending from the proximal end to the distal end. The body may include an outer surface and an inner surface defining a lumen of the body. The flow restriction device may further include a fluid pathway disposed between the distal end and the proximal end when the outer surface is coupled within a mating luer. The fluid pathway may include a non-linear portion on at least a portion of the outer surface along which a fluid flows from the distal end into the fluid collection device.

### CROSS-REFERENCE TO RELATED APPLICATIONS

This application claims the benefit of U.S. Provisional Application No. 63/239,639, titled “PIVC-INTEGRATED HEMOLYSIS-REDUCTION ACCESSORIES FOR DIRECT BLOOD DRAW,” filed on Sep. 1, 2021, the disclosure of which is incorporated herein by reference in its entirety.

### TECHNICAL FIELD

The present disclosure generally relates to blood draw and administration of parenteral fluids to a patient, and particularly to systems and methods to reduce hemolysis in PIVC blood draw.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used as an internal vacuum or a vacuum tube. The blood collection container may also be a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container.

The description provided in the background section should not be assumed to be prior art merely because it is mentioned in or associated with the background section. The background section may include information that describes one or more aspects of the subject technology.

### SUMMARY

In accordance with various embodiments of the present disclosure, a flow restriction device may include a distal end configured to couple to a catheter assembly, a proximal end including a proximal connector configured to couple to a fluid collection device, a body extending from the proximal connector to the distal end, and a fluid pathway disposed between the distal end and the proximal end when the outer surface is coupled within a mating luer. The body may include an outer surface and an inner surface defining a lumen of the body. The fluid pathway may have a non-linear portion on at least a portion of the outer surface along which a fluid flows from the distal end into the fluid collection device.

In accordance with various embodiments of the present disclosure, a blood collection system may include a blood collection device, and a flow restriction device fluidly coupled to the blood collection device. The flow restriction device may include an outer surface, and an inner surface defining an internal flowpath therethrough, a distal end including a distal connector configured to couple to a catheter assembly, and a proximal end coupled to the blood collection device and having a lumen fluidly communicated with the internal flowpath. The flow restriction device may further include a non-linear fluid pathway disposed on the outer surface at least partially between the distal end and the proximal end when the outer surface is coupled within a mating luer.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1A illustrates a peripheral intravenous catheter (PIVC) assembly that includes a flow restriction device, in accordance with some embodiments of the present disclosure.

FIG. 1B illustrates a vascular access device including a peripheral intravenous catheter (PIVC) assembly having a flow restriction device and a blood collection device, in accordance with some embodiments of the present disclosure.

FIG. 1C is an operational illustration of a vascular access device including a peripheral intravenous catheter (PIVC) assembly having a flow restriction device and a blood collection device, in accordance with some embodiments of the present disclosure.

FIG. 1D is an operational illustration of an infusion of peripheral intravenous catheter (PIVC) assembly that includes a flow restriction device and a blood collection device, in accordance with some embodiments of the present disclosure.

FIG. 2A illustrates a vascular access device including a peripheral intravenous catheter (PIVC) assembly having a flow restriction device, in accordance with some embodiments of the present disclosure.

FIG. 2B illustrates a vascular access device including a peripheral intravenous catheter (PIVC) assembly having a flow restriction device with a check valve and coupled to a needleless connector, in accordance with some embodiments of the present disclosure.

FIG. 3A illustrates a vascular access device including a peripheral intravenous catheter (PIVC) assembly having a flow restriction device with a vent plug disposed therein, in accordance with some embodiments of the present disclosure.

FIG. 3B illustrates a vascular access device including a peripheral intravenous catheter (PIVC) assembly having a flow restriction device with a check valve and a vent plug disposed therein, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a flow restriction device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a flow restriction device) that regulates the overall flow rate of the entire fluid path as blood cells travel through. The flow restriction device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

In some embodiments, the flow restriction device may integrate a check valve in the central fluid path that allows hemolysis reduction and unobstructed infusion.

According to various embodiments of the present disclosure, the flow restriction device may be an insert with luer access and spiral continuous channel on the exterior featuring the flow resistance per design. The flow restriction device may be a plastic insert, as molded, featuring the fluid channel of flow resistance per design. The proximal end of the insert may have a female luer so that a vent plug may be inserted into the flow restriction device that is connected to a port of a luer adapter of the PIVC as packaged to enable blood flashback when the PIVC is placed. In some embodiments, a clinician or other user may remove the vent plug from the flow restriction device and attach a blood collection device to complete the blood draw. In some embodiments, when the clinician connects a blood collection device to this insert, the vent plug may be pushed into a pocket and thereby open the fluid path for blood draw.

Accordingly, the flow restriction devices and systems of the various embodiments described herein are advantageous in that the spiral continuous channel with small (minimized) diameter may increase a length of the fluid pathway defined by the continuous channel or groove through which the blood flows, and may provide increased flow resistance and decreased blood flow rate within the flow resistance device in contrast to the linear internal fluid pathways. As such, a risk of hemolysis during blood collection may be advantageously be reduced.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Furthermore, the flow restriction devices have the potential to stay inline throughout PIVC indwell for multiple blood draws. Additionally, since the flow restriction devices are an add-on which can be easily incorporated without any changes to existing PIVC, there is minimal impact to clinical setting and operations.

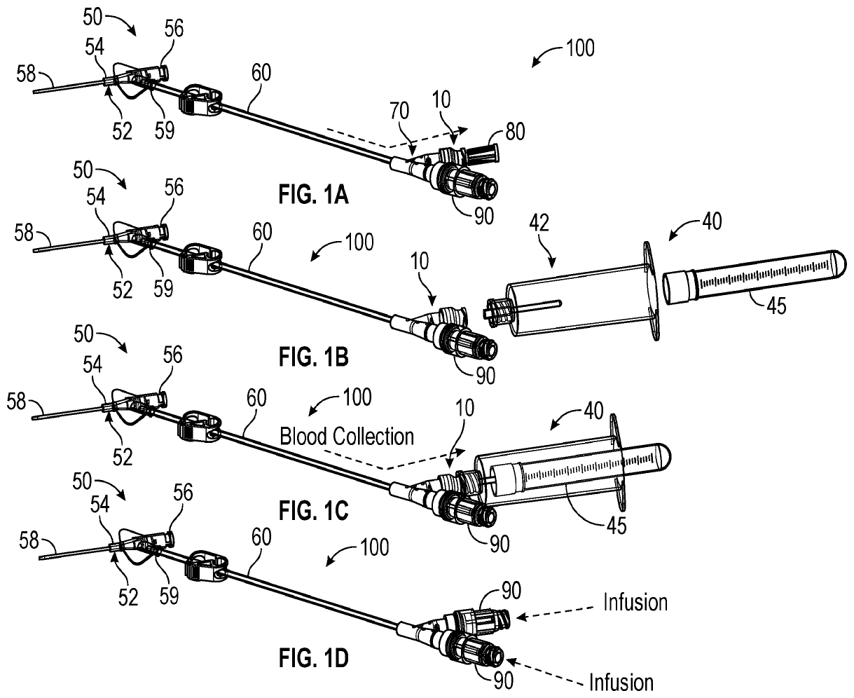

FIGS. 1A-1D illustrate a vascular access device 100 including a peripheral intravenous catheter (PIVC) assembly 50 that includes a flow restriction device 10, in accordance with some embodiments of the present disclosure.

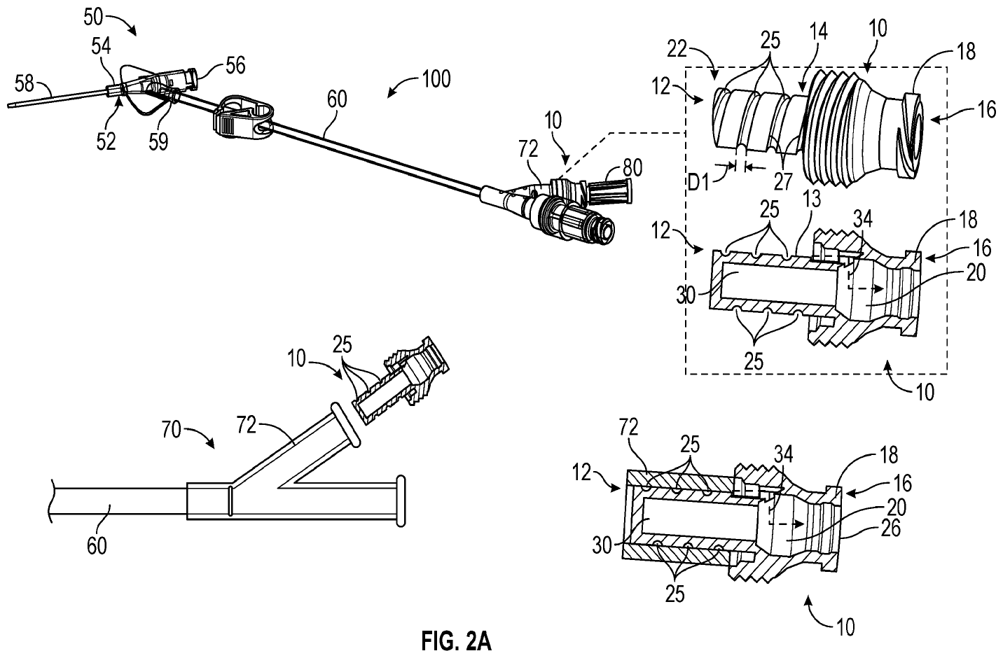

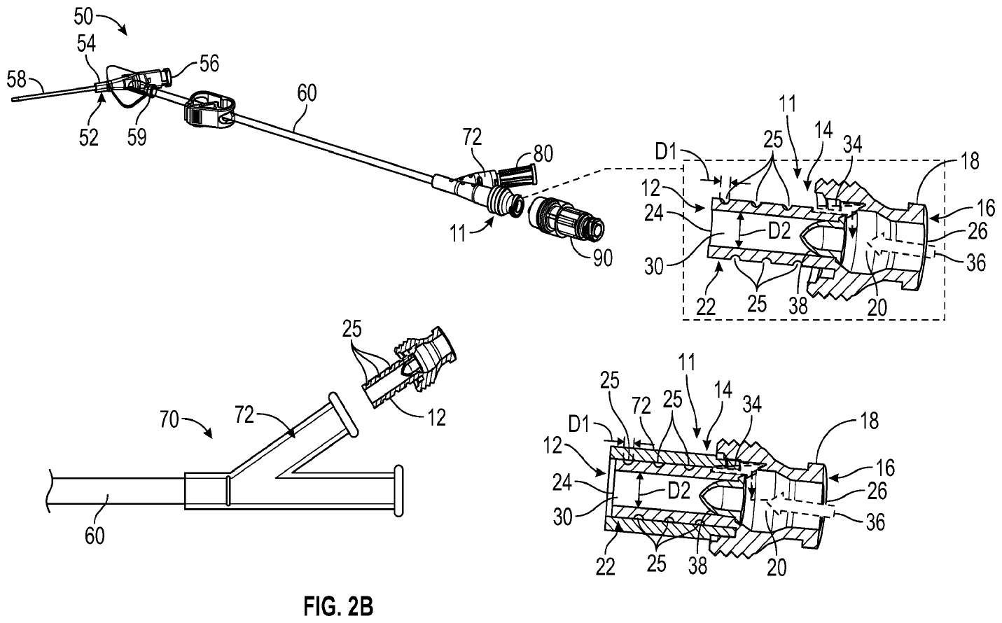

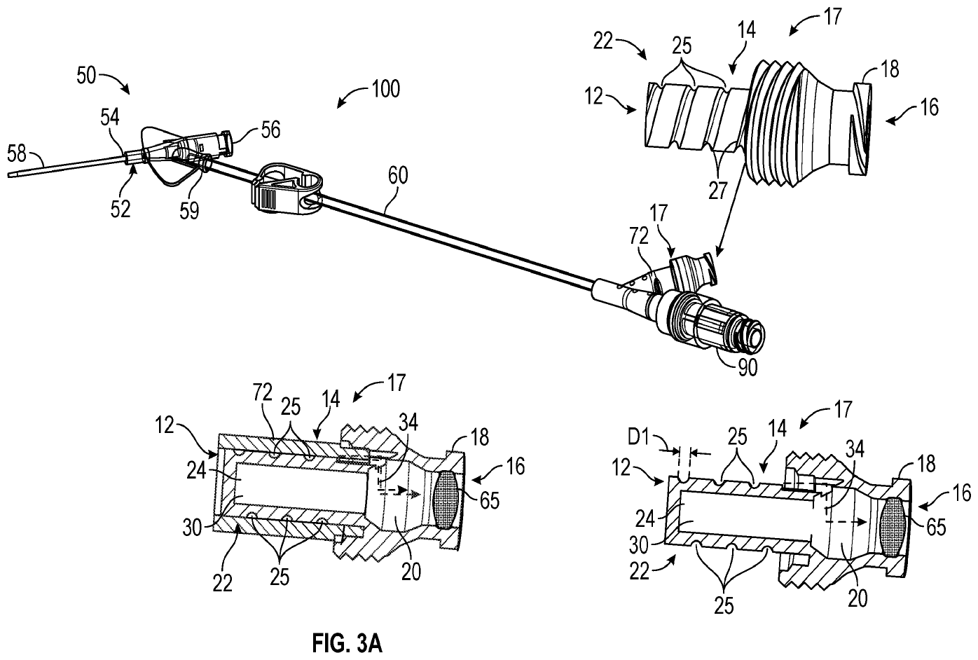

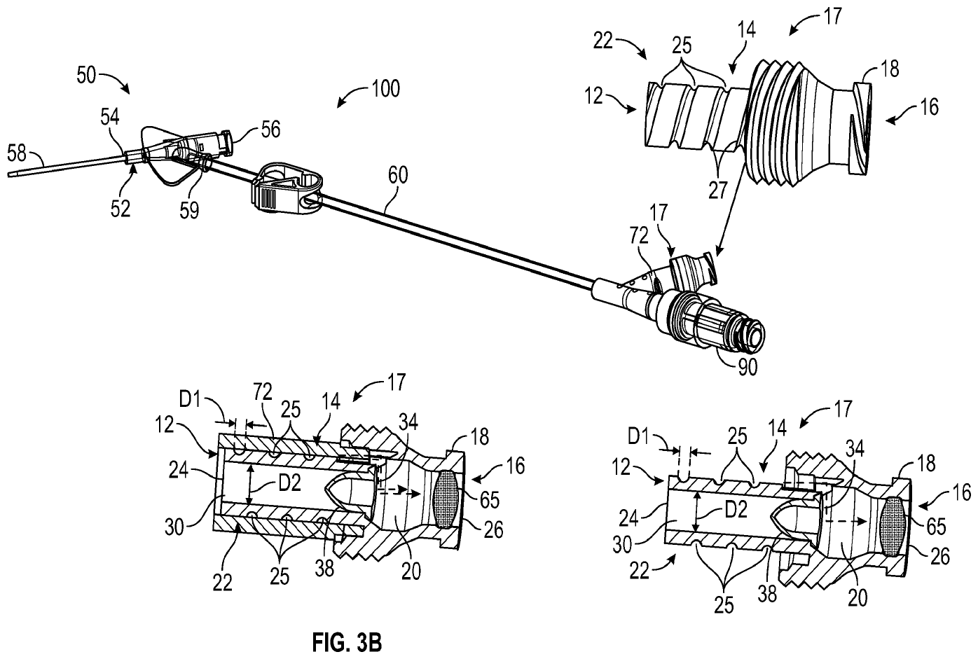

Referring now to FIGS. 1A-1C, flow restriction device 10 is illustrated, according to some embodiments. The flow restriction device 10 may be configured to reduce a likelihood of hemolysis during blood collection using a vascular access device 100. In some embodiments, the vascular access device 100 may include a catheter assembly (e.g., a PIVC) 50. In some embodiments, (as further illustrated in FIGS. 2A-3B), the flow restriction device 10 may include a distal end 12, which may include a body or distal connector 14 configured to couple to the catheter assembly 50. The distal connector 14 may include a male luer connector, or another suitable connector.

In some embodiments, the catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter 70, such as, for example, a Y-adapter or single port luer adapter. In some embodiments, the distal connector 14 of flow restriction device 10 may be configured to couple to the Y-adapter 70.

In some embodiments, the catheter assembly 50 may be non-integrated and may not include the extension tube 60. In these and other embodiments, the flow restriction device 10 may be configured to couple to the proximal end 56 of the catheter hub 52 or another suitable portion of the catheter assembly 50. In some embodiments, the catheter assembly 50 may be coupled to a removable extension tube 60. In some embodiments, the flow restriction device 10 may be coupled directly to the catheter adapter, eliminating the extension tube and providing a compact catheter system.

FIG. 2A illustrates a vascular access device 100 including a peripheral intravenous catheter (PIVC) assembly that includes a flow restriction device 10, in accordance with some embodiments of the present disclosure. As illustrated in FIG. 2A, with continued reference to FIGS. 1B and 1C, in some embodiments, the flow restriction device 10 may include a proximal end 16, which may include a proximal connector 18 configured to couple to a blood collection device 40 (illustrated in FIG. 1). In some embodiments, the proximal end 16 may be coupled to the blood collection device 40. For example, the proximal end 16 may be integrated with the blood collection device 40 or monolithically formed with the blood collection device 40 as a single unit. As another example, the proximal end 16 may include a female luer connector, which may be coupled with a male luer connector of the blood collection device 40.

In some embodiments, the proximal connector 18 may be a female luer connector or another suitable connector. The proximal connector 18 may include a lumen 20 extending therethrough for coupling to a male luer portion of the blood collection device. In some embodiments, the distal connector 14 of the flow restrictor device 10 may be in the form of a cylindrical body extending from the proximal connector 18 to the distal end 12. As depicted, a fluid pathway may be disposed on at least a portion of an outer surface 13 between the distal end 12 and the proximal end 16. In some embodiments, the fluid pathway may include a non-linear portion 22 on at least a portion of the outer surface 13. The fluid pathway may be configured such that a medical fluid 34 flows from the distal end 12 and into fluid collection device 40 via the proximal connector 18. For example, where blood is being withdrawn or collected from a patient, the medical fluid 34 may be a blood sample, and the fluid collection device 40 may be a blood collection device. In some embodiments, the blood collection device may be a luer lock access device (LLAD). Accordingly, during blood collection or withdrawal from the patients, the blood sample 34 may flow along the outer surface 13 on the non-linear portion 22 from the distal end 12 into the LLAD 40.

In particular, as depicted the non-linear portion 22 of the fluid pathway may include a continuous channel or groove 25. In some embodiments, the non-linear portion 22 may form a coil shape, an S-shape, or another suitable non-linear, winding shape. For example, the continuous channel or groove 25 may have a coil shape (which may include a spiral) recessed in the outer surface 13 of the non-linear portion 22. In some embodiments, the continuous channel or groove 25 may have an S-shape recessed in the outer surface 13 of the non-linear portion 22. Where the medical fluid 34 is blood being withdrawn from a patient, blood cells may experience shear stress as they flow from the distal end 12 to the proximal end 16 of the flow restriction device 10. For example, the maximum shear stress may be along the wall of the blood cell, often referred to as wall shear stress. Wall shear stress on blood cells is considered a major source of mechanical damage to blood cells causing hemolysis of the blood cells. In some embodiments, the non-linear portion 22 may facilitate increased flow resistance within the vascular access system 100 to distribute the pressure differential and reduce shear stress experienced by the red blood cells of the blood 34.

According to various embodiments of the present disclosure, no fluid flowing through the non-linear portion 22 may flow in a straight line. In some embodiments, the non-linear portion 22 may advantageously increase a length of the fluid pathway defined by continuous channel or groove 25 through which the blood flows. The minimized diameter of the fluid pathway defined by continuous channel or groove 25 may provide increased resistance to flow of the blood 34 and thereby decrease blood flow rate within the flow resistance device 10. Since the decreased blood flow rate causes a reduction in shear stress experienced by the red blood cells of the blood 34, a risk of hemolysis during blood collection may advantageously be reduced.

In some embodiments, a leg 72 of the Y-adapter 70 may form an outer component for coupling with the non-linear portion 22 of the flow restriction device 10. For example, the leg 72 of Y-adapter 70 may include a lumen into which the outer surface 13 of the non-linear portion 22 having the continuous channel or groove 25 may be coupled. Accordingly, the non-linear portion 22 may form an inner component having the continuous channel or groove 25 which may be coupled in the lumen of the outer component (the leg 72 of Y-adapter 70). In some embodiments, the continuous channel or groove 25 may include a coil or spiral shape. In some embodiments, the continuous channel or groove 25 may be surrounded, encased, or otherwise enveloped in the lumen of the leg 72 of Y-adapter 70, which may bound the continuous channel or groove 25 such that fluid flowing through the continuous channel or groove 25 may not escape the continuous channel or groove 25 except at a distal end and a proximal end of the continuous channel or groove 25. Accordingly, the continuous channel or groove 25 when encased, inserted or otherwise enveloped in the lumen of the leg 72 of Y-adapter 70 may define the non-linear fluid pathway through which fluid (e.g., blood) flows from the distal end 12 to the proximal end 16 for collection in the fluid collection device 40.

As depicted, coupling and/or contact between the inner component (i.e., the non-linear portion 22) and the outer component (the leg 72 of Y-adapter 70) may form a seal between the non-linear portion 22 and the leg 72 of Y-adapter 70. In some embodiments, the outer surface of the non-linear portion 22 may include a seal element 27, which may include silicon, rubber, plastic, or another suitable material. In some embodiments, the seal element 27 may have a coil or spiral shape and may be offset from the continuous channel or groove 25 in the distal-proximal direction. The seal element 27 may prevent fluid from escaping the continuous channel or groove 25 except at a distal end and a proximal end of the continuous channel or groove 25.

According to various embodiments of the present disclosure, an outer diameter of the non-linear portion 22 may be approximately equal to or slightly less than an inner diameter of the lumen of the leg 72 of Y-adapter 70 such that the non-linear portion 22 is fitted within the lumen of the leg 72 of Y-adapter 70. For example, in some embodiments, the non-linear portion 22 and the leg 72 of Y-adapter 70 may form a male luer to female luer connection. For example, the outer surface of the non-linear portion 22 having the continuous channel or groove 25 may be encased, enveloped, or otherwise coupled in the lumen of the leg 72 of Y-adapter 70.

In some embodiments, the lumen of the leg 72 of Y-adapter 70 may be generally cylindrical, and the outer surface 13 of the non-linear portion 22 may be generally cylindrical. In some embodiments, the lumen of the leg 72 of Y-adapter 70 may be a standard female luer, and the outer surface 13 of the non-linear portion 22 may be a standard male luer. In some embodiments, the non-linear portion 22 and the leg 72 may be concentric. In some embodiments, the non-linear portion 22 and the leg 72 may be integrally formed or monolithically formed as a single unit.

According to various embodiments, a proximal end of the continuous channel or groove 25 may include a hole that may fluidly connect the continuous channel or groove 25 to an opening of the proximal end 16. Similarly, in some embodiments, a distal end of the continuous channel or groove 25 may include a hole that may fluidly connect the continuous channel or groove 25 to an opening of the distal end 12.

In some embodiments, a removable vent plug 80 may be coupled to the lumen 20 at the proximal end 16 of the flow resistance device 10. The vent plug 80 may allow air to vent from the catheter assembly 50 during the catheter insertion process. This may advantageously allow blood to flashback to fill the fluid path from the catheter through the adapter 56 and the extension tube 60 to provide the clinician with visual confirmation that the catheter has been properly placed within a patient's vein. Accordingly, blood may fill the catheter system all the way up to the vent plug 65 that may allow air to escape, but prevent blood leakage. The vent plug 80 may be removed prior to connecting the blood collection device 40.

In some embodiments, the flow resistance device 10 may include a vent plug (similar to the vent plug 65 illustrated in FIG. 3A below) disposed in the lumen 20 at the proximal end 16. The vent plug 65 may be incorporated for non-obstructed extended flashback on the catheter assembly 50. As shall be described in further detail below with respect to FIG. 3A, the vent plug 65 may allow air to vent from the catheter assembly 50 during the catheter insertion process. This may advantageously allow blood to flashback to fill the fluid path from the catheter through the adapter 56 and the extension tube 60 to provide the clinician with visual confirmation that the catheter has been properly placed within a patient's vein.

FIG. 2B illustrates a peripheral intravenous catheter (PIVC) assembly that includes a flow restriction device 11 having a check valve 38 and coupled to a needleless connector 90, in accordance with some embodiments of the present disclosure. Referring now to FIG. 2B, a flow restriction device 11 is illustrated, according to some embodiments. In some embodiments, the flow restriction device 11 may be similar or identical in terms of one or more included features and/or operation to the flow restriction device 10. For example, the flow restriction device 11 may be coupled to the blood collection device 40 (see, for example, FIG. 1C). According to various embodiments of the present disclosure, the body 14 may include the outer surface 13 and an inner surface 15 defining a lumen 30 of the body 14. The lumen 30 of the body 14 and the lumen 20 of the proximal connector 18 may be selectively fluidly communicated via the check valve 38. When fluidly communicated, the lumen 30 of the body 14 and the lumen 20 of the proximal connector 18 may form an internal flowpath 24 in which a fluid, e.g., an intravenous (IV) fluid 36 flows from the lumen 20 at the proximal end 16 to the distal end 12 and into the catheter assembly 50. In these embodiments, the fluid, e.g., IV fluid 36 may flow from the lumen 20 at the proximal end 16 to the distal end 12 and into the catheter assembly 50 via the continuous channel or groove 25 and the hole that fluidly connects the continuous channel or groove 25 to an opening of the distal end 12. Accordingly, the fluid, e.g. IV fluid may flow from the proximal end 16 to the distal end 12 via both the internal flowpath 24 and the continuous channel or groove 25, and into the catheter assembly 50.

In some embodiments, the internal flowpath 24 in which the IV fluid 36 flows from the proximal end 16 to the distal end 12 and into the catheter assembly 50 is a linear flowpath. Accordingly, fluid flowing through the internal flowpath 24 may flow linearly. In some embodiments, the linearity of internal flowpath 24 through which the IV fluid 36 flows may advantageously result in a decreased length of the flowpath 24 as compared to a curved or spiraling flowpath, and may thereby decrease flow resistance and increase IV fluid flow rate to the patient within the flow resistance device 11. Accordingly, the IV fluid 36 may flow uninterrupted or un-delayed to the patient whilst still reducing the risk of hemolysis during blood collection.

Similar to the flow restriction device 10, the flow restriction device 11 may include a fluid pathway disposed on at least a portion of the outer surface 13 between the distal end 12 and the proximal end 16. In some embodiments, the fluid pathway may include the non-linear portion 22 on at least a portion of the outer surface 13. Similar to the flow restriction device 10, the non-linear portion 22 of the fluid pathway of fluid restriction device 11 may include a continuous channel or groove 25. In some embodiments, the non-linear portion 22 may form a coil shape, an S-shape, or another suitable non-linear, winding shape. For example, the continuous channel or groove 25 may have a coil shape (which may include a spiral) recessed in the outer surface 13 of the non-linear portion 22. In some embodiments, the continuous channel or groove 25 may have an S-shape recessed in the outer surface 13 of the non-linear portion 22. Where the medical fluid 34 is blood being withdrawn from a patient, blood cells may experience shear stress as they flow from the distal end 12 to the proximal end 16 of the flow restriction device 11. In some embodiments, the non-linear portion 22 may facilitate increased flow resistance within the vascular access system 100 to distribute the pressure differential and reduce shear stress experienced by the red blood cells of the blood 34.

Additionally, in some embodiments, the continuous channel or groove 25 of the non-linear portion 22 may have a first diameter D1, the internal flowpath 24 within the lumen 30 may have a second diameter D2, and the second diameter D2 may be larger than the first diameter D1. The aforementioned configuration with the second diameter D2 being larger than the first diameter D1 may be advantageous in further allowing an unrestricted and increased amount of the IV fluid 36 to flow to the patient versus the smaller (minimized) diameter D1. Accordingly, the IV fluid 36 may flow in a second unrestricted (less flow resistance) direction (proximal to distal) opposite from the first direction (distal to proximal) in which the blood sample 34 having blood cells flows.

According to various embodiments, the check valve 38 may disposed in the internal flowpath 24. The check valve 38 may be configured to prevent the medical fluid (e.g., blood sample 34) from flowing from the distal end 12 to the proximal end 16 via the internal flowpath 24. The check valve 38, may however allow the IV fluid 36 to flow from the proximal end 16 to the distal end 12. Accordingly, the blood sample 34 containing blood cells may be forced to flow through the curved or spiral continuous channel or groove 25 of the non-linear portion 22 in order to arrive at the blood collection device 40, while the IV fluid 36 may flow to the catheter assembly 50 through the internal flowpath 24 and the curved or spiral continuous channel or groove 25.

The aforementioned configuration is advantageous in that the spiral continuous channel or groove 25 of non-linear portion 22—by virtue of its wrapping around the outer surface 13—may increase a length of the fluid pathway defined by continuous channel or groove 25 through which the blood sample 34 flows. Further, due to the minimized diameter or size of the fluid pathway defined by continuous channel or groove 25, the fluid pathway defined by continuous channel or groove 25 may thereby provide increased flow resistance and decreased blood flow rate within the flow resistance device 11 in contrast to the linear internal flowpath 24. Accordingly, a risk of hemolysis during blood collection may advantageously be reduced.

FIG. 3A illustrates a peripheral intravenous catheter (PIVC) assembly that includes a flow restriction device having a vent plug 65 disposed therein, in accordance with some embodiments of the present disclosure. Referring now to FIG. 3A, a flow restriction device 17 is illustrated, according to some embodiments. In some embodiments, the flow restriction device 17 may be similar or identical in terms of one or more included features and/or operation to the flow restriction device 10. For example, the flow restriction device 17 may be coupled to the blood collection device 40 (see, for example, FIG. 1C). As depicted, the flow restriction device 17 may include a vent plug 65 disposed in the lumen 20 at the proximal end 16. The vent plug 65 may be incorporated for non-obstructed extended flashback on the catheter assembly 50.

In some embodiments, the vent plug 65 may allow air to vent from the catheter assembly 50 during the catheter insertion process. This may advantageously allow blood to flashback to fill the fluid path from the catheter through the adapter 56 and the extension tube 60 to provide the clinician with visual confirmation that the catheter has been properly placed within a patient's vein. Accordingly, blood may fill the catheter system all the way up to the vent plug 65 that may allow air to escape, but prevent blood leakage. The clinician may then connect the blood collection device 40. During connection of the blood connection device 40, the male luer of the blood collection device 40 may push the vent plug 65 from its position at the proximal end 16 and further into the lumen 20 of the flow restriction device 10, thereby opening a fluid pathway for blood collection. Accordingly, the internal vent plug 65 may advantageously serve the same purpose as the removable vent plug 80 illustrated in FIG. 2A. The internal vent plug 65 may serve a similar purpose as the vent plug 80 with the difference between the vent plug 65 and the vent plug 80 being that the vent plug 80 may be removed by the clinician prior to connecting the blood collection device 40, while vent plug 65 may be pushed out of place by connection of the blood collection device 40.

In some embodiments, the fluid within entering the lumen 20 from the fluid pathway in the non-linear portion 22 may flow through the proximal end 16 in response to an axial displacement of the vent plug 65 disposed within the proximal end 16. For example, in some embodiments, the vent plug 65 may be displaced and open the fluid pathway in response to coupling of the blood collection device 40 to the proximal end 16 of the flow restriction device 17. Accordingly, the vent plug 65 may be removed/nudged to open the fluid pathway 20 at the proximal end 16 for blood to travel into from the curved or spiral continuous channel or groove 25 into the blood collection device 40.

Accordingly, in operation the blood sample 34 containing blood cells may be forced to flow through the curved or spiral continuous channel or groove 25 of the non-linear portion 22 in order to arrive at the blood collection device 40. The aforementioned configuration is advantageous in that the spiral continuous channel or groove 25 of non-linear portion 22 may increase a length of the fluid pathway defined by continuous channel or groove 25 through which the blood flows, and may thereby provide increased flow resistance and decreased blood flow rate within the flow resistance device 17 in contrast to a linear fluid pathway. Accordingly, a risk of hemolysis during blood collection may be advantageously be reduced.

FIG. 3B illustrates a vascular access device including a peripheral intravenous catheter (PIVC) assembly having a flow restriction device 19 with a check valve and a vent plug disposed therein, in accordance with some embodiments of the present disclosure. In some embodiments, the flow restriction device 19 may be similar or identical in terms of one or more included features and/or operation to the flow restriction device 11, with the addition of the vent plug 65 disposed in the lumen 20 at the proximal end 16. The vent plug 65 may be incorporated for non-obstructed extended flashback on the catheter assembly 50. The vent plug 65 may be similar in structure, function and purpose as the vent plug 65 described above with respect to FIG. 3A, therefore a detailed description thereof shall be omitted with respect to FIG. 3B.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Furthermore, the flow restriction devices have the potential to stay inline throughout PIVC indwell for multiple blood draws. Additionally, since the flow restriction devices are an add-on which can be easily incorporated without any changes to existing PIVC, there is minimal impact to clinical setting and operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction device, comprising: a distal end configured to couple to a catheter assembly; a proximal end including a proximal connector configured to couple to a fluid collection device; a body extending from the proximal connector to the distal end, the body comprising an outer surface and an inner surface defining a lumen of the body; and a fluid pathway disposed between the distal end and the proximal end when the outer surface is coupled within a mating luer, the fluid pathway comprising a non-linear portion on at least a portion of the outer surface along which a fluid flows from the distal end into the fluid collection device.

Clause 2. The flow restriction device of Clause 1, wherein the non-linear portion comprises a continuous groove having a coil shape recessed in the outer surface.

Clause 3. The flow restriction device of Clause 1, wherein the non-linear portion comprises a continuous groove having an S-shape recessed in the outer surface.

Clause 4. The flow restriction device of Clause 1, wherein the fluid flowing from the distal end into the fluid collection device comprises blood and the fluid collection device comprises a blood collection device.

Clause 5. The flow restriction device of Clause 4, wherein the blood collection device comprises a luer lock access device.

Clause 6. The flow restriction device of Clause 1, wherein the lumen of the body defines an internal flowpath in which a fluid flows from the proximal end to the distal end and into the catheter assembly.

Clause 7. The flow restriction device of Clause 6, wherein the fluid flowing from the proximal end to the distal end and into the catheter assembly comprises an intravenous (IV) fluid.

Clause 8. The flow restriction device of Clause 6, wherein the internal flowpath in which a fluid flows from the proximal end to the distal end and into the catheter assembly comprises a linear flowpath.

Clause 9. The flow restriction device of Clause 6, wherein the non-linear portion of the fluid pathway comprises a first diameter, the internal flowpath comprises a second diameter, and the second diameter is larger than the first diameter.

Clause 10. The flow restriction device of Clause 6, further comprising a check valve disposed in the internal flowpath, the check valve configured to (i) prevent fluid flowing from the distal end to the proximal end via the internal flowpath, and (ii) allow fluid to flow from the proximal end to the distal end via the internal flowpath.

Clause 11. The flow restriction device of Clause 1, further comprising a vent plug disposed in the lumen at the proximal end.

Clause 12. A blood collection system, comprising: a blood collection device; and a flow restriction device fluidly coupled to the blood collection device, the flow restriction device comprising: an outer surface, and an inner surface defining an internal flowpath therethrough; a distal end, comprising a distal connector configured to couple to a catheter assembly; a proximal end coupled to the blood collection device and comprising a lumen fluidly communicated with the internal flowpath; and a non-linear fluid pathway disposed on the outer surface at least partially between the distal end and the proximal end when the outer surface is coupled within a mating luer.

Clause 13. The blood collection system of Clause 12, wherein the blood collection device comprises a luer lock access device.

Clause 14. The blood collection system of Clause 12, wherein the non-linear fluid pathway comprises a continuous a groove having a coil shape recessed in the outer surface.

Clause 15. The blood collection system of Clause 12, wherein the non-linear fluid pathway comprises a continuous groove having an S-shape recessed in the outer surface.

Clause 16. The blood collection system of Clause 12, wherein the distal connector comprises a male luer connector.

Clause 17. The blood collection system of Clause 12, wherein the non-linear fluid pathway comprises a first diameter, the internal flowpath comprises a second diameter, and the second diameter is larger than the first diameter.

Clause 18. The blood collection system of Clause 12, further comprising a check valve disposed in the internal flowpath, the check valve configured to (i) prevent fluid flowing from the distal end into the proximal end, and (ii) allow fluid to flow from the proximal end to the distal end.

Clause 19. The blood collection system of Clause 12, further comprising a vent plug disposed in the lumen of the proximal end.

Clause 20. The blood collection system of Clause 12, wherein the proximal end comprises a proximal connector, wherein the proximal connector comprises a female luer connector.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flow restriction device, comprising:
a distal end configured to couple to a catheter assembly;
a proximal end including a proximal connector configured to couple to a fluid collection device;
a body extending from the proximal connector to the distal end, the body comprising an outer surface and an inner surface defining a lumen of the body; and
a fluid pathway disposed between the distal end and the proximal end when the outer surface is coupled within a mating luer, the fluid pathway comprising a non-linear portion on at least a portion of the outer surface along which a fluid flows from the distal end into the fluid collection device.

2. The flow restriction device of claim 1, wherein the non-linear portion comprises a continuous groove having a coil shape recessed in the outer surface.

3. The flow restriction device of claim 1, wherein the non-linear portion comprises a continuous groove having an S-shape recessed in the outer surface.

4. The flow restriction device of claim 1, wherein the fluid flowing from the distal end into the fluid collection device comprises blood and the fluid collection device comprises a blood collection device.

5. The flow restriction device of claim 4, wherein the blood collection device comprises a luer lock access device.

6. The flow restriction device of claim 1, wherein the lumen of the body defines an internal flowpath in which a fluid flows from the proximal end to the distal end and into the catheter assembly.

7. The flow restriction device of claim 6, wherein the fluid flowing from the proximal end to the distal end and into the catheter assembly comprises an intravenous (IV) fluid.

8. The flow restriction device of claim 6, wherein the internal flowpath in which a fluid flows from the proximal end to the distal end and into the catheter assembly comprises a linear flowpath.

9. The flow restriction device of claim 6, wherein the non-linear portion of the fluid pathway comprises a first diameter, the internal flowpath comprises a second diameter, and the second diameter is larger than the first diameter.

10. The flow restriction device of claim 6, further comprising a check valve disposed in the internal flowpath, the check valve configured to (i) prevent fluid flowing from the distal end to the proximal end via the internal flowpath, and (ii) allow fluid to flow from the proximal end to the distal end via the internal flowpath.

11. The flow restriction device of claim 1, further comprising a vent plug disposed in the lumen at the proximal end.

12. A blood collection system, comprising:
a blood collection device; and
a flow restriction device fluidly coupled to the blood collection device, the flow restriction device comprising:
an outer surface, and an inner surface defining an internal flowpath therethrough;
a distal end, comprising a distal connector configured to couple to a catheter assembly;
a proximal end coupled to the blood collection device and comprising a lumen fluidly communicated with the internal flowpath; and
a non-linear fluid pathway disposed on the outer surface at least partially between the distal end and the proximal end when the outer surface is coupled within a mating luer.

13. The blood collection system of claim 12, wherein the blood collection device comprises a luer lock access device.

14. The blood collection system of claim 12, wherein the non-linear fluid pathway comprises a continuous a groove having a coil shape recessed in the outer surface.

15. The blood collection system of claim 12, wherein the non-linear fluid pathway comprises a continuous groove having an S-shape recessed in the outer surface.

16. The blood collection system of claim 12, wherein the distal connector comprises a male luer connector.

17. The blood collection system of claim 12, wherein the non-linear fluid pathway comprises a first diameter, the internal flowpath comprises a second diameter, and the second diameter is larger than the first diameter.

18. The blood collection system of claim 12, further comprising a check valve disposed in the internal flowpath, the check valve configured to (i) prevent fluid flowing from the distal end into the proximal end, and (ii) allow fluid to flow from the proximal end to the distal end.

19. The blood collection system of claim 12, further comprising a vent plug disposed in the lumen of the proximal end.

20. The blood collection system of claim 12, wherein the proximal end comprises a proximal connector, wherein the proximal connector comprises a female luer connector.
